(19) United States
(12) Patent Application Publication
(10) Pub. No.: US 2021/0283180 A1
(43) Pub. Date: Sep. 16, 2021

(54) CAR T CELL THERAPY TO TARGET T CELL SPECIFIC CANCERS

(71) Applicant: The Arizona Board of Regents on Behalf of The University of Arizona, Tucson, AZ (US)

(72) Inventor: Michael Worobey, Tucson, AZ (US)

(21) Appl. No.: 17/258,062

(22) PCT Filed: Jul. 3, 2019
(86) PCT No.: PCT/US19/40551

§ 371 Date: Jan. 5, 2021

Related U.S. Application Data
(60) Provisional application No. 62/694,797, filed on Jul. 6, 2018.

(51) Int. Cl. A61K 35/17; C12N 5/0783; C07K 14/725; C07K 14/705

(52) U.S. Cl. CPC A61K 35/17; C12N 5/0636; C07K 14/7051; A61K 2039/5156; C07K 14/70578; C07K 2319/30; C07K 2319/33; C07K 14/70521

(57) ABSTRACT

Disclosed are chimeric antigen receptor (CAR) polypeptides comprising a T cell receptor (TCR) antigen binding domain, a transmembrane domain, and an intracellular signaling domain. Disclosed are methods of making a CAR T cell comprising obtaining a cell from a subject diagnosed with T cell lymphoma; determining the sequence of the TCR on the cell; and transducing a T cell with a vector comprising a nucleic acid sequence that encodes a CAR polypeptide, wherein the CAR polypeptide comprises a TCR antigen binding domain, a transmembrane domain, and an intracellular signaling domain, wherein the TCR antigen binding domain is specific to a subsequence of the sequence of the TCR on the cell identified in the step of determining the sequence of the TCR

### BACKGROUND

An emerging cancer therapy, Chimeric Antigen Receptor (CAR) T cell therapy removes a patient's T cells using apheresis, genetically modifies the patient's T cells to recognize and attack cancer cells bearing a generic cell surface receptor present of the patient's tumor cells (e.g. CD19), then infuses the modified T cells back into the patient. Such “CD19 CAR T” therapy is limited to cancers (e.g. some B cell lymphomas) which express that protein. Another CD19 CAR T limitation is that non-cancer cells also express CD19 and so are killed as collateral damage. The present invention, custom CAR T cells, overcomes this limitation by modifying a patient's T cells to attack a specific sequence on the T cell receptor (TCR) of the patient's tumor cell. This approach provides a more selective means to target only tumor cells within a patient rather than tumor cells plus non-tumor cells bearing generic cell surface antigens like CD19.

### BRIEF SUMMARY

Disclosed are chimeric antigen receptor (CAR) polypeptides comprising a T cell receptor (TCR) antigen binding domain, a transmembrane domain, and an intracellular signaling domain.

Disclosed are nucleic acid sequences capable of encoding a CAR polypeptide comprising a TCR antigen binding domain, a transmembrane domain, and an intracellular signaling domain.

Disclosed are vectors comprising the nucleic acid sequence of the disclosed CAR nucleic acid sequences.

Disclosed are cells comprising any of the disclosed CAR polypeptides, CAR nucleic acids, or disclosed vectors.

Disclosed are methods of making a CAR T cell comprising obtaining a tumor cell from a subject diagnosed with a disease or disorder involving undesired proliferation of T cells; determining the sequence of the TCR on the tumor cell; and transducing a T cell with a vector comprising a nucleic acid sequence that encodes a CAR polypeptide, wherein the CAR polypeptide comprises a TCR antigen binding domain, a transmembrane domain, and an intracellular signaling domain, wherein the TCR antigen binding domain is specific to a subsequence of the sequence of the TCR on the tumor cell identified in the step of determining the sequence of the TCR.

Disclosed are methods of treating T cell lymphoma comprising administering an effective amount of a T cell genetically modified to express a CAR polypeptide comprising a TCR antigen binding domain, a hinge and transmembrane domain, and an intracellular signaling domain.

Also disclosed are methods of treating a disease or disorder involving undesired proliferation of T cells comprising administering an effective amount of a vector comprising the nucleic acid sequence capable of encoding a disclosed CAR polypeptide to a subject in need thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combination and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a CAR polypeptide” includes a plurality of such CAR polypeptides, reference to “the CAR polypeptide” is a reference to one or more CAR polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range¬ from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

A “single-chain variable fragment (scFv)” means a protein comprising the variable regions of the heavy and light chains of an antibody. A scFv can be a fusion protein comprising a variable heavy chain, a linker, and a variable light chain.

An “antibody antigen-binding fragment (Fab)” is a region of an antibody that binds to antigen. An Fab comprises constant and variable regions from both heavy and light chains.

An “antigen-binding fragment” is a peptide that binds to the antigen of interest. For example, an antigen-binding fragment can be a peptide that binds to a TCR. In some instances, an antigen-binding fragment can be a peptide aptamer. In some instances an antigen-binding fragment can be a peptide to which an oligonucleotide aptamer with suitable antigen binding properties is covalently or non-covalently conjugated. In some aspects, an antigen-binding fragment can be a peptide derived via phage display biopanning.

The term “patient specific” means anything that is specific to a single subject or patient, not a population or subpopulation of patients. For example, a composition such as a protein, nucleic acid or compound can be “patient specific” in that it binds to a specific sequence found only in one individual, subject or patient. In some aspects, in order to determine if something is patient specific (e.g. a patient specific TCR), a sequence of the patient must first be determined (e.g. sequenced) and a peptide, protein, nucleic acid, or compound must be identified that specifically binds to the patient specific target (e.g TCR). For example, an antibody that simply binds to a cancer marker, such as CD19 or HER2, is not patient specific because that antibody binds to an entire population or subpopulation of patients that are CD19+ or HER2+. A general target such as CD19+ or HER2+ are population specific not patient specific. In some aspects, the disclosed TCR binders or TCR binding domains are patient specific as they bind to a specific TCR sequence of a single patient or individual that is not found in another individual.

A “patient specific TCR” or “subject specific TCR” as used herein refers to a TCR sequence unique to the patient or subject. A “patient specific TCR” or “subject specific TCR” as used herein refers to a TCR sequence of a single patient or individual that is not found in another individual.

A “patient specific TCR binder” or “subject specific TCR binder” as used herein refers to a composition such as a peptide, protein, nucleic acid or compound that specifically binds to or hybridizes to a patient specific TCR or subject specific TCR. A “patient specific TCR binder” or “subject specific TCR binder” as used herein refers to a composition that specifically binds to or hybridizes to a TCR sequence of a single patient or individual that is not found in another individual. In some aspects, a “patient specific TCR binder” or “subject specific TCR binder” as used herein refers to a composition that has the same or similar attributes and specificity as a TCR antigen binding domain.

### B. Chimeric Antigen Receptor (CAR) Polypeptides

Disclosed are chimeric antigen receptor (CAR) polypeptides comprising a T cell receptor (TCR) antigen binding domain, a transmembrane domain, and an intracellular signaling domain.

The TCR antigen binding domain, transmembrane domain, and intracellular signaling domain can be any of those described herein and any combination of those described herein.

In some instances, any of the disclosed CAR polypeptides can further comprise a tag sequence. In some instances, the tag sequence can be located between the TCR antigen binding domain and the transmembrane domain or between the TCR antigen binding domain and a hinge region. In some instances, the tag sequence can be a hemagglutinin tag, histidine tag, glutathione-S-transferase tag, or fluorescent tag. For example, the tag can be any sequence capable of aiding in the purification of the CAR polypeptide or capable of detecting the CAR polypeptide.

### 1. T Cell Receptor (TCR) Antigen Binding Domain

In some instances, the TCR antigen binding domain can be an antibody fragment or an antigen-binding fragment that specifically binds to the TCR. In some instances, the TCR antigen binding domain can be any recombinant or engineered protein domain capable of binding the TCR. In some instances, the TCR antigen binding domain binds to a specific TCR variant. In some instances, a specific TCR variant comprises at least one mutation, insertion, or deletion in the TCR. The at least one mutation, insertion, or deletion in the TCR can be present in complementarity region 1 (CDR1), complementarity region 2 (CDR2), complementarity region 3 (CDR3), or hypervariable region 4 of the TCR.

In some instances, the TCR antigen binding domain can be a Fab, a single-chain variable fragment (scFv) of an antibody, an antigen binding peptide, or an aptamer that specifically binds the TCR. In some instances, the scFv, comprising both the heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some instances, the TCR antigen binding domain can comprise a heavy chain variable region, a light chain variable region, and a linker that links the heavy chain variable region to the light chain variable region. In some instances, the linker can be directly involved in the binding of the TCR to the TCR antigen binding domain. In some instances, the linker can be indirectly involved in the binding of the TCR to the TCR antigen binding domain.

### 2. Transmembrane Domain

In some instances, the transmembrane domain comprises an immunoglobulin Fc domain. In some instances, the immunoglobulin Fc domain can be an immunoglobulin G Fc domain.

In some instances, the transmembrane domain comprises a CD8α domain, CD3ζ, FcεR1γ, CD4, CD7, CD28, OX40, or H2-Kb.

In some instances, the transmembrane domain can be located between the TCR antigen binding domain and the intracellular signaling domain.

### 3. Intracellular Signaling Domain

In some instances, the intracellular signaling domain comprises a co-stimulatory signaling region. In some instances, the co-stimulatory signaling region can comprise the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some instances, the intracellular signaling domain can be a T cell signaling domain. For example, the intracellular signaling domain can comprise a CD3ζ signaling domain. In some instances, CD3ζ signaling domain is the intracellular domain of CD3ζ.

In some instances, the intracellular signaling domain comprises a CD3ζ signaling domain and a co-stimulatory signaling region, wherein the co-stimulatory signaling region comprises the cytoplasmic domain of CD28, 4-1BB, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some aspects, the disclosed CAR polypeptides can comprise more than one intracellular signaling domain. For example, a CAR polypeptide can comprise more than one co-stimulatory signaling region.

### 4. Hinge Region

Any of the disclosed CAR polypeptides can further comprise a hinge region. For example, disclosed are CAR polypeptides comprising a TCR antigen binding domain, a transmembrane domain, and an intracellular signaling domain and further comprising a hinge region.

In some instances, the hinge region can be located between the TCR antigen binding domain and the transmembrane domain.

In some instances, the hinge region allows for the TCR antigen binding domain to bind to the antigen. For example, the hinge region can increase the distance of the binding domain to the cell surface and provide flexibility.

### 5. Additional Element

Any of the disclosed CAR polypeptides can further comprise an additional element. An additional element can be a controllable on-off switch or a molecule to enhance T cell function, enrichment, and minimize senescence.

A controllable on-off switch can allow for the CAR polypeptide to be turned off when negative side effects occur. In some aspects, turning the CAR polypeptide off results in the cell undergoing apoptosis to avoid further side effects. In some aspects, the CAR polypeptide can be turned on and off by administering a particular drug or compound to the cell having the CAR polypeptide.

### C. TCR Binder

Disclosed are TCR binders. A TCR binder is a peptide, nucleic acid, or compound that binds to a patient specific TCR. For example, in some aspects, a TCR binder can be an antibody or antigen binding fragment of an antibody that binds to a patient specific TCR.

In some aspects, a TCR binder is determined by sequencing a TCR, making peptide sequences identical to at least a portion of the sequences determined by sequencing a TCR, performing a hybridization assay, such as phage display, with a library of molecules to determine which molecules bind to the peptide sequences identical to at least a portion of the sequences determined by sequencing a TCR, wherein a molecule that binds to the peptide sequences identical to at least a portion of the sequences determined by sequencing a TCR is a TCR binder.

In some aspects, a TCR binder is conjugated to a tag. A tag is a molecule that can be recognized and specifically bound by a tag binding domain. In some aspects, a tag binding domain is part of a universal CAR polypeptide. Exemplary tags include, but are not limited to, fluorescein isothiocyanate (FITC), dinitrophenol, peridinin chlorophyll protein complex, green fluorescent protein, biotin, phycoerythrin (PE), histidine, streptavidin, biotin, avidin, horse radish peroxidase, palmitoylation, nitrosylation, alkalanine phosphatase, glucose oxidase, Glutathione S-transferase, maltose binding protein, part of a leucine zipper, and any types of fluorescent materials including quantum dot nanocrystals. In some aspects a tag can be a random peptide.

In some aspects, a TCR binder is directly or indirectly conjugated to a tag. For example, indirect conjugation can involve a linker in between the TCR binder and the tag.

In some aspects, one or more TCR binders can be conjugated to a carrier. In some aspects, a carrier can be a nanoparticle. For example, a nanoparticle can be, but is not limited to, a carbon-based nanoparticle, a ceramic nanoparticle, a metal-based nanoparticle (e.g. gold nanoparticle, iron oxide nanoparticle), a polymeric nanoparticle (e.g. polyethylene glycol-based nanoparticle), a lipid-based nanoparticle, a dendrimers, a liposome, or a magnetic nanoparticle. Thus, in some aspects, a tag is conjugated to the carrier.

Disclosed are compositions comprising a TCR binder and a tag. In some aspects, the tag is conjugated to the TCR binder. In some aspects, the tag and the TCR binder are both conjugated to a carrier. Disclosed are compositions comprising a subject specific TCR binder and a tag, wherein the subject specific TCR binder will bind specifically to a TCR of the subject.

### D. Universal CAR Polypeptides

Disclosed are universal CAR polypeptides that comprise any of the transmembrane domains, intracellular signaling domains, and hinge regions disclosed throughout. In some aspects, a universal CAR polypeptide comprises any of the elements disclosed throughout except a TCR antigen binding domain. In other words, in some aspects a universal CAR polypeptide can comprise any of the transmembrane domains, intracellular signaling domains, hinge regions, and/or additional elements disclosed throughout.

In some aspects, a universal CAR polypeptide further comprises a tag binding domain. In some aspects, a tag binding domain binds to a tag. For example, a tag can be conjugated to a TCR binder or a carrier conjugated to a TCR binder. Thus, the binding of a tag binding domain, present on the universal CAR polypeptide, to a tag conjugated to a TCR binder completes the immunological synapse and leads to the killing of the T cell comprising the TCR that the TCR binder is specific to.

### E. Tag Binding Domain

This tag binding domain is typically present at the amino terminal end of the polypeptide of the T cell genetically modified to express a universal CAR polypeptide. Locating the tag-binding domain at the amino terminus permits the tag-binding domain unfettered access to the tag of a TCR binder. In some aspects, the tag-binding domain can be fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein or a composition that binds to fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkalanine phosphatase, glucose oxidase, and maltose binding protein.

In some aspects the tag binding domain binds to a random peptide used as the tag. Thus, the tag binding domain can be determined by performing phage display to identify something that binds to the random peptide tag.

In some aspects, the tag-binding domain can be a nucleic acid, protein, peptide, or antibody or an antigen-binding fragment thereof.

In some aspects the tag binding domain is an antibody or fragment. The identity of the antibody or fragment is only limited by the identity of the tag of the tagged protein. For example, the antibodies may be obtained from a phage display library or from any species of animal, though preferably from a mammal such as a human, simian, mouse, rat, rabbit, guinea pig, horse, cow, sheep, goat, pig, dog or cat. Preferably the antibodies are human or humanized antibodies. Nor is there a limitation on the particular class of antibody that may be used, including IgG1, IgG2, IgG3, IgG4, IgM, IgA1, IgA2, IgD and IgE antibodies. Antibody fragments include single-chain variable fragment (scFv), single chain antibodies, F(ab′)2 fragments, Fab fragments, and fragments produced by an Fab expression library, with the only limitation being that the antibody fragments retain the ability to bind the selected tag.

The antibodies may also be polyclonal, monoclonal, or chimeric antibodies, such as where an antigen binding region (e.g., F(ab′)2 or hypervariable region) of a non-human antibody is transferred into the framework of a human antibody by recombinant DNA techniques to produce a substantially human molecule. Antigen-binding fragments, such as scFv, may be prepared therefrom.

In one aspect, the tag binding domain is a single-chain variable fragment (scFv). A scFv comprises the variable regions of the heavy (VH) and light chains (VL) of an antibody, typically linked via a short peptide of ten to about 25 amino acids. The linker can either connect the N-terminus of the VH with the C-terminus of the VL, or vice versa.

The binding specificity of the tag-binding domain will depend on the identity of the tag of the TCR binder. For example, when the tag is FITC (Fluorescein isothiocyanate), the tag-binding domain may constitute an anti-FITC scFv. Alternatively, when the tag is biotin the tag-binding domain may constitute an anti-biotin scFv or a natural biotin-binding molecule including streptavidin or avidin. When the tag is PE (phycoerythrin), the tag-binding domain may constitute an anti-PE scFv.

### F. CAR Nucleic Acid Sequences

Disclosed are nucleic acid sequences capable of encoding any of the disclosed CAR polypeptides or universal CAR polypeptides. For example, disclosed are nucleic acid sequences capable of encoding a CAR polypeptide comprising a TCR antigen binding domain, a transmembrane domain, and an intracellular signaling domain. Also disclosed are nucleic acid sequences capable of encoding a universal CAR polypeptide comprising a tag binding domain, a transmembrane domain, and an intracellular signaling domain.

### G. Vectors

Disclosed are vectors comprising the nucleic acid sequence of the disclosed CAR nucleic acid sequences. In some instances, the vector can be selected from the group consisting of a DNA, a RNA, a plasmid, and a viral vector. In some instances, the vector can comprise a promoter.

### H. Cells

Disclosed are cells comprising any of the disclosed CAR polypeptides, universal CAR polypeptides, CAR nucleic acids, or disclosed vectors. These cells can be considered genetically modified.

In some instances, the cell can be, but is not limited to, T cells or NK cells. In some instances, the T cell can be a γδ T cell or an αβ T cell. For example, T cells can be a CD8+ T cell and NK cells can be NK-92 cells. In some instances, the cell can be a human cell.

Thus, disclosed are T cells and NK cells expressing one of the CAR polypeptides disclosed herein.

### I. Phage Display Library

Disclosed are phage display libraries comprising antigen-binding peptides or scFvs that bind to TCRs. In some instances, the library displays scFv domains comprising both heavy and light chain variables. In some instances, the library displays antibodies comprising a TCR antigen binding domain. In some instances, the library displays antigen-binding peptides comprising a TCR antigen binding domain. In some instances, the library displays antibodies comprising a TCR antigen binding domain for an individual subject.

### J. Methods of Making

Disclosed are methods of making a CAR T cell comprising obtaining a tumor cell from a subject diagnosed with a disease or disorder involving undesired proliferation of T cells; determining the sequence of the TCR on the tumor cell; and transducing a T cell with a vector comprising a nucleic acid sequence that encodes a CAR polypeptide, wherein the CAR polypeptide comprises a TCR antigen binding domain, a transmembrane domain, and an intracellular signaling domain, wherein the TCR antigen binding domain is specific to a subsequence of the sequence of the TCR on the tumor cell identified in the step of determining the sequence of the TCR.

In some instances, a disease or disorder involving undesired proliferation of T cells can include, but is not limited to, T cell lymphomas, T lymphocytic leukemias, non-Hodgkin lymphomas, graft-versus-host disease (GVHD), HIV/AIDS, rheumatoid arthritis, multiple sclerosis, ME/CFS, celiac disease, and autoimmune lymphoproliferative syndrome.

As described herein, the CAR polypeptide can comprise any of the elements disclosed herein. In some instances, the TCR antigen binding domain can be an antibody fragment or an antigen-binding fragment that specifically binds to the TCR. Thus, once the sequence of the TCR on the tumor cell has been determined, a peptide with that specific sequence can be used to produce an antigen-binding peptide or antibody, such as a monoclonal antibody, and the antibody can then be used to make the antibody fragment that can be the TCR antigen binding domain.

Disclosed are methods of making a cell comprising transducing a T cell with any of the disclosed vectors.

A method of making a CAR T cell comprising obtaining a cell from a subject diagnosed with a disease or disorder involving undesired proliferation of T cells; determining the sequence of the TCR on the cell; and transducing a T cell with a vector comprising a nucleic acid sequence that encodes a CAR polypeptide, wherein the CAR polypeptide comprises a TCR antigen binding domain, a transmembrane domain, and an intracellular signaling domain, wherein the TCR antigen binding domain is specific to a subsequence of the sequence of the TCR on the cell identified in step b).

### K. Methods of Treating

Disclosed are methods of treating a disease or disorder involving undesired proliferation of T cells comprising administering an effective amount of a T cell genetically modified to express one or more of the disclosed CAR polypeptides to a subject in need thereof. For example, disclosed are methods of treating a disease or disorder involving undesired proliferation of T cells comprising administering an effective amount of a T cell genetically modified to express a CAR polypeptide comprising a TCR antigen binding domain, a hinge and transmembrane domain, and an intracellular signaling domain.

Disclosed are methods of treating a disease or disorder involving undesired proliferation of T cells comprising administering an effective amount of a T cell genetically modified to express one or more of the disclosed universal CAR polypeptides to a subject in need thereof. In some aspects the subject in need thereof is further administered a composition comprising a TCR binder and a tag. In some aspects, the tag is conjugated to the TCR binder or the tag and the TCR binder are both conjugated to a carrier. In some aspects, the composition comprising a TCR binder and a tag is administered to a subject in need thereof after the subject has been administered a T cell genetically modified to express one or more of the disclosed universal CAR polypeptides. In some aspects, the composition comprising a TCR binder and a tag is administered to a subject in need thereof simultaneously with the subject has been administered a T cell genetically modified to express one or more of the disclosed universal CAR polypeptides. In some aspects, the composition comprising a TCR binder and a tag is administered to a subject in need thereof prior to the subject has been administered a T cell genetically modified to express one or more of the disclosed universal CAR polypeptides.

Disclosed are methods of treating T cell lymphoma comprising administering an effective amount of a T cell genetically modified to express a universal CAR polypeptide comprising a tag binding domain, a transmembrane domains, an intracellular signaling domains, and a hinge region to a subject in need thereof and administering a composition comprising a subject specific TCR binder and a tag to the subject in need thereof.

Disclosed are methods of treating a subject diagnosed with a disease or disorder involving undesired proliferation of T cells comprising administering an effective amount of a T cell genetically modified to express a universal CAR polypeptide comprising a tag binding domain, a transmembrane domains, an intracellular signaling domains, and a hinge region to a subject in need thereof and administering a composition comprising a subject specific TCR binder and a tag to the subject in need thereof.

In some aspects of the disclosed methods, the composition comprising a TCR binder and a tag is administered to the subject after the T cell genetically modified to express a universal CAR polypeptide is administered to the subject.

In some aspects of the disclosed methods, the TCR binder binds to a subject specific TCR. In some aspects, the subject specific TCR binder is determined by sequencing a TCR on a cell obtained from the subject and determining a sequence that will bind to a subject specific TCR.

Disclosed are methods of treating a disease or disorder involving undesired proliferation of T cells comprising administering an effective amount of at least one of the disclosed vectors to a subject in need thereof. For example, disclosed are methods of treating a disease or disorder involving undesired proliferation of T cells comprising administering an effective amount of a vector comprising the nucleic acid sequence capable of encoding a disclosed CAR polypeptide to a subject in need thereof. In some instances, the vectors can comprise targeting moieties. In some instances, the targeting moieties target T cells.

In some instances, the disclosed methods of treating a disease or disorder involving undesired proliferation of T cells further comprise administering a therapeutic agent. In some instances, the therapeutic agent can be, but is not limited to, conventional chemotherapy, vaccines, monoclonal antibodies, T cell immunotherapies, and other immunomodulatory agents. In some instances, the therapeutic agent can be proteasome inhibitors, immunomodulatory agents, histone deacetylase inhibitors, monoclonal antibodies, bispecific antibodies, or immune checkpoint inhibitors.

In some instances, a disease or disorder involving undesired proliferation of T cells can include, but is not limited to, T cell lymphomas, T lymphocytic leukemias, non-hodgkin lymphomas, graft-versus-host disease (GVHD), HIV/AIDS, rheumatoid arthritis, multiple sclerosis, ME/CFS, celiac disease, and autoimmune lymphoproliferative syndrome.

Also disclosed are methods of killing T cell lymphoma cells in a subject comprising administering an effective amount of a T cell genetically modified to express one or more of the disclosed CAR polypeptides to a sample comprising T cell lymphoma cells, wherein the TCR antigen binding domain of the CAR polypeptide is specific to the T cell receptor sequence present on the subject's T cell lymphoma cells.

In some aspects, when administering to a subject in need thereof a T cell genetically modified to express one or more of the disclosed universal CAR polypeptides and a composition comprising a TCR binder and a tag, the composition comprising a TCR binder and tag can be titrated based on the CAR T cell response seen in the subject in need thereof. For example, a subject in need thereof that has been administered a T cell genetically modified to express one or more of the disclosed universal CAR polypeptides can receive low concentrations of a composition comprising a TCR binder and tag. Once it has been determined that CAR T cell activity is low, increased concentrations of a composition comprising a TCR binder and tag can be administered. If, upon administration of a composition comprising a TCR binder and tag, to a subject that has been administered a T cell genetically modified to express one or more of the disclosed universal CAR polypeptides, the subject experiences negative side effects then the concentration of the composition comprising a TCR binder and tag can be reduced until side effects subside.

### L. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the disclosed CAR polypeptides, universal CAR polypeptides or TCR binders. In some instances, the kits can contain any of the disclosed vectors.

### EXAMPLES

### A. T Cells and T cell Receptor Diversity

T lymphocytes (T cells) bind to antigens through their T cell receptor complex. TCRs are highly polymorphic between T cell clones. The chains of the TCR of a T cell clone are each composed of a unique combination of domains known as variable “V”, diversity “(D)”, joining “J”, and constant “C” (Chien et al., 1984, Nature 312:31-35). There are multiple genomic loci coding for different variants of these domains (for the beta chain of the TCR, for example, there are >50 possible V domains and a smaller number of (D) and J domains). The combination, via somatic recombination, of different V, (D) and J domains (in “alpha” and “beta” chains in >90% of T cells, or “delta” and “gamma” chains in a minority), along with additional insertions or deletions at the junctions of domains, leads to a virtually unlimited amount of possible TCR diversity and unique binding specificities for each T cell clone. This specificity, mediated by the unique TCR of each T cell clone, allows customized anti-TCR antigen-binding peptides or antibodies to differentiate between these specificities and provides a means to create customized CAR T cells as discussed in detail below.

Several important diseases and disorders involve the undesirable proliferation of particular T cells clones. For example, peripheral T cell lymphomas (PTCLs) are non-Hodgkin lymphomas typically constituting a single, malignant T cell clone. Graft versus host disease (GVHD) involves T cells from a stem cell transplant donor that proliferate in an uncontrolled fashion in the transplant recipient. And HIV-1 involves latently-infected resting memory CD4+ T cells than contain proviral HIV-1 genomes integrated within host DNA. These latently-infected cells proliferate, copying the virus genome as they do so, and such proliferating T cell clones are a major reason that HIV-1 is currently incurable. In each of these illnesses, just one or a few T cell clones tend to be involved either throughout the entire course of the disease or at a given time point during the disease. Highly selective destruction of targeted T cell clones would provide game-changing therapeutic advances.

To date, immunotherapies that aim to target T cell-caused diseases including PTCL have seen limited success. Cell-surface antigens like CD19 that can effectively discriminate diseased (e.g. malignant) cells from healthy cells, and which have been targeted for other diseases (including B cell lymphomas), are typically not present on T cells. And approaches based on more generic antigens present on many or all T cells are prohibitively toxic since ablation of much or all of a patient's T cell population leads to loss of cellular immunity and to cytokine-release syndrome.

An experimental CAR T cell therapy approach based on an anti-TCR antibody that binds to one, but not the other, of the two different TCR beta chain C domains has been proposed (Maciocia et al., 2017, Nature Medicine 2017 23:1416-1423). However, this approach would still lead to loss of approximately 35% to 65% of healthy T cells in a patient and would be likely to lead to dangerous effects of off-target cell killing, such deficiencies in cellular immunity and cytokine-release syndrome.

### B. TCR Clonotypic CAR T Cell Therapy

The current invention exploits the extensive diversity of TCR repertoires, such that no two T cell clones within an individual are likely to have identical TCRs and TCR binding specificities. Clonotypic anti-TCR antigen-binding peptides or antibodies are generated for each T cell clone and are used as the antigenic receptor domain in CAR T cells. In a patient with PTCL, for example, where only a single malignant clone is usually present, patient samples are collected by biopsy from affected tissues during a period when malignant T cells are present, usually at the time initial diagnosis is made (e.g. via lymph node excision or core needle biopsy, or bone marrow aspirate, with tissues then frozen or fixed in formalin and embedded in paraffin). The TCR gene sequences coding for all or part of the TCR are characterized by sequencing the DNA of the rearranged (somatically recombined) TCR genes of that clone or by sequencing messenger RNA (mRNA) of the transcribed region, for example using PCR or RT-PCR with probes and primers specific to TCR loci, followed by Sanger sequencing or Next Generation sequencing.

A target peptide of at least 8 amino acids in length, based on all or a portion of the V, D (if present; only in beta chains), J, and C domain, is synthesized and used as the immunogen to generate a monoclonal antibody (for example in BALB/c mice), or as the antigen in a phage display assay to discover a Fab, scFv,antigen-binding peptide, or aptamer with suitable binding properties. For TCR beta chains, this target peptide may include CDR1, CDR2, CDR3, or HV4 or may be a concatenation of two or more of these regions.

A library can be created based on a wide sampling of known TCRs that comprises two to hundreds or thousands of antigen-binding peptides or scFv sequences. Newly inferred TCR sequences can be screened against the library of antigen-binding peptides or scFv sequences to determine if there is a best fit. This allows for a pre-defined antigen binding domain to be used instead of having to make a new antigen-binding peptide or monoclonal antibody for each patient. An antigen-binding peptide or scFv library can be generated using phage display that will bind to TCRs with >95% specificity, and sufficient affinity and t_{1/2} to work as CARs. In other words, for a given TCR there is a clonotypic antigen-binding peptide or scFv that will bind with high affinity and half-life to the TCR but will bind <5% of the remaining TCR repertoire in that patient.

The monoclonal antibody, Fab, scFv, antigen-binding peptide, or aptamer derived from this sequence is then tested for binding affinity against the TCR of the target T cell clone (in this example the malignant PTCL clone) and against a large array of healthy T cells from the same individual, for example using surface plasmon resonance, flow cytometry, ELISA, or confocal microscopy. The anti-TCR antibody is tested to establish both high affinity (e.g. K_{D}<500 nM) and half-life (e.g. >20 minutes) for the target TCR and low affinity (>100 times lower) for all or the vast majority (e.g. 95%) of non-target T cells. This sequence is then used to encode the CAR polypeptide and to generate a patient- and clone-specific therapy with low or non-existent off-target T cell killing and limiting unwanted side-effects such as cytokine-release syndrome, while maintaining cellular immunity. Cellular immunity is confirmed by recovering T cells specific for particular viruses through incubation of peripheral blood mononuclear cells (PBMCs) with pools of antigenic peptides.

### C. Universal CARs

The main challenge of a universal CAR T approach to targeting T cells in T cell lymphoma/leukemia and other diseases involving unwanted proliferation of certain clonal T cell populations is in developing bispecific adapter molecules or nanoparticles the can establish an immunological synapse between the target T cell and the CAR T cell. There are two components of the bispecific adapter molecules or nanoparticles—a component that binds the target T cells' TCR with high specificity and sensitivity, which we call the ‘TCR-binder’, and a component that is bound with high binding affinity by the antigen binding domain of the chimeric antigen receptor, which we call the ‘tag’. The TCR binder binds specifically and sensitively to the target T cell clone, while the CAR T cell binds to the tag, completing the immunological synapse and triggering CAR T cell killing of the target T cell.

Of these two components, the main challenge is in developing a TCR binder that is specific to the T cell clone one is trying to target; the choice of tag and the matching antigen-binding domain of the CAR T cell is trivial, and several such systems have already been developed (e.g. a FITC tag and a scFv CAR from a monoclonal antibody raised to bind FITC, or a biotin tag and an avidin or strepavidin CAR, etc.). In some aspect, a small peptide tag can be synthesized that is not expressed on human cells and a CAR can be developed that binds to it (i.e. the tag binding domain of the CAR can bind to the small peptide tag).

Disclosed is a system that can allow one to move quickly, in a matter of weeks, from sequencing the target T cell TCR to developing a TCR binder for use in an adapter molecule in a universal CAR T approach.

The TCR beta chain CDR3 of a widely used T cell leukemia cell line called Jurkat has been sequenced. The CDR3 is the most variable region within TCR beta, the region that gives the T cell clone its specificity and which is most closely involved in binding to the antigen epitopes displayed by MHC molecules under natural conditions. To rapidly develop TCR binders that bind with high sensitivity and specificity to this region of the TCR peptides, called ‘target peptides’, were synthesized that represent either the entire Jurkat TCR beta CDR3 or a portion of it, labelling the N-terminus with biotin:

Peptide 1: Biotin-CASSFSTCSANYGYTF

Peptide 3: Biotin-SASSFSTPSANYGYTF

Peptide 5: Biotin-ASSFSTPSANYGYT

These peptide were synthesized using solid state chemistry and were purified to >95% (Lifetein, LLC, Hillsborough, N.J.). These target peptides were then used as the target molecules in phage display biopanning assays using both a nine-amino acid phage display peptide library (Ph.D. 7 Phage Display Peptide Library Kit, New England Biolabs) or a 12-amino acid peptide library (Phage Display Peptide Library Kit, New England Biolabs). The kit manual protocols were modified for use of biotin-labeled target peptides rather than peptides immobilized to a plate. Three rounds of phage amplification and positive selection for peptides with high affinity for the target peptide were conducted (using streptavidin-coated magnetic beads and magnet separation). One round of negative selection to remove peptides that bind to streptavidin was also conducted.

TCR binders recovered from the phage display biopanning assays were then cloned and sequenced (20 to 40 for each target peptide). In each case, multiple identical putative TCR binders were recovered for each target peptide, and some of these peptides were also recovered across different biopanning assays using the different Jurkat CDR3 target peptides.

To determine whether putative TCR binders recovered by biopanning are actually specific for Jurkat TCR beta CDR3, bioinformatic approaches were used to determine whether any of the putative TCR binders were target-unrelated peptides (for example, peptides known the have high growth rates or to bind to streptavidin). None were.

Next an ELISA assay was used to determine whether the most commonly recovered putative TCR binders from the phage display assays for each CDR3 target peptide actually bind the target peptide. Biotinylated CDR3 target peptide was immobilized on streptavidin-coated polysterene 96-well plates, wells were blocked with biotin, add biotinylated putative TCR binders were added, plates were washed, then streptavidin-HRP was added. If the putative TCR binders actually bind to the target peptides, an enzymatic reaction of streptavidin-HRP leads to a detectable color change mediated by streptavidin-HRP bound to the biotin of the TCR binder, which is, in turn, bound to the CDR3 target peptide bound to the plate.

For each CDR3 target peptide, the most common putative TCR binding peptide raised by phage display biopanning gave a strong ELISA result, indicating that the putative TCR binder does bind the CDR3 peptide. Moreover, the TCR binders raised for each variant of the Jurkat TCR beta CDR3 also bound the other variants of CDR3.

These TCR binders can then be tested for strongly binding to Jurkat CDR3 in its native configuration in TCR of live Jurkat cells.

A key point of this approach is that it is very fast. One can move from TCR sequence to a TCR binder in a matter of weeks, which is fast enough for clinical feasibility. The TCR binders can be used in a multivalent form by conjugating several copies to a single nanoparticle to increase binding avidity to the T cell being targeted. These nanoparticles would also be decorated with tag molecules specific for the universal CAR T cells. Therefore, for an actual patient, the only thing that needs to be customized is the TCR binder of the adapter molecule or nanoparticle.

Those skilled in the art will recognize, or be able to ascertain using no more than routine experimentation, many equivalents to the specific embodiments of the method and compositions described herein. Such equivalents are intended to be encompassed by the following claims.

1. A method of making a CAR T cell comprising:
a) obtaining a cell from a subject diagnosed with a disease or disorder involving undesired proliferation of T cells;
b) determining the sequence of the TCR on the cell; and
c) transducing a T cell with a vector comprising a nucleic acid sequence that encodes a CAR polypeptide, wherein the CAR polypeptide comprises a TCR antigen binding domain, a transmembrane domain, and an intracellular signaling domain, wherein the TCR antigen binding domain is specific to a subsequence of the sequence of the TCR on the cell identified in step b).

2. The method of claim 1, wherein the intracellular signaling domain comprises a co-stimulatory signaling region.

3. The method of claim 2, wherein the co-stimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

4. The method of claim 1, wherein the intracellular signaling domain is a T cell signaling domain.

5. The method of claim 4, wherein the intracellular signaling domain comprises a CD3 zeta (CD3ζ) signaling domain.

6. The method of claim 1, wherein the intracellular signaling domain comprises a CD3ζ signaling domain and a co-stimulatory signaling region, wherein the co-stimulatory signaling region comprises the cytoplasmic domain of CD28 or 4-1BB.

7. The method of claim 1, wherein the TCR antigen binding domain is an antibody, antibody fragment, an antigen-binding fragment, a Fab, a single-chain variable fragment (scFv) of an antibody, antigen-binding peptide, or an aptamer that specifically binds to the subsequence of the sequence of the TCR.

8. (canceled)

9. The method of claim 1, wherein the transmembrane domain comprises an immunoglobulin Fc domain.

10. (canceled)

11. The method of claim 1, wherein the transmembrane domain comprises a CD8α domain, CD3ζ, FcεR1γ, CD4, CD7, CD28, OX40, or H2-Kb.

12. The method of claim 1, wherein the transmembrane domain is located between the TCR antigen binding domain and the intracellular signaling domain.

13. The method of claim 1 further comprising a tag sequence.

14. The method of claim 13, wherein the tag sequence is located between the TCR antigen binding domain and the transmembrane domain.

15. (canceled)

16. The method of claim 1 further comprising a hinge region.

17. The method of claim 16, wherein the hinge region is located between the TCR antigen binding domain and the transmembrane domain.

18. The method of claim 1, wherein the disease or disorder involving undesired proliferation of T cells is T cell lymphoma or T lymphocytic leukemia.

19. The method of claim 1, wherein the cell is a tumor cell.

20. A chimeric antigen receptor (CAR) polypeptide, comprising a T cell receptor (TCR) antigen binding domain, a transmembrane domain, and an intracellular signaling domain.

21.-44. (canceled)

45. A T cell expressing the CAR polypeptide of claim 20.

46.-49. (canceled)

50. A method of killing T cell lymphoma cells in a subject comprising administering an effective amount of a T cell genetically modified to express a CAR polypeptide of claim 20 to a sample comprising T cell lymphoma cells, wherein the T cell receptor (TCR) antigen binding domain of the CAR polypeptide is specific to the T cell receptor sequence present on the subject's T cell lymphoma cells.

51.-55. (canceled)

56. A method of treating a subject diagnosed with a disease or disorder involving undesired proliferation of T cells comprising:
a. administering an effective amount of a T cell genetically modified to express a universal CAR polypeptide comprising a tag binding domain, a transmembrane domains, an intracellular signaling domains, and a hinge region to a subject in need thereof; and
b. administering a composition comprising a subject specific TCR binder and a tag to the subject in need thereof.

57. (canceled)
